(19) United States
(12) Patent Application Publication
(10) Pub. No.: US 2008/0292495 A1
(43) Pub. Date: Nov. 27, 2008

(54) Method and device for the continuous reduction of the odor contamination caused by the waste water in sewers and method and device for determining the pollution of a water sample with odorous substances

(75) Inventors: Franz-Bernd Frechen, Kassel (DE); Michaela Frey, Kassel (DE); Marco Ohme, Oberlistingen (DE); Sebastian Grahlow, Naumburg (DE)

Correspondence Address: PYLE & PIONTEK;ATTN: THOMAS R. VIGIL, 221 N LASALLE STREET , ROOM 2036, ROOM 2036, CHICAGO, IL, 60601, US

(73) Assignee: Universitat Kassel, Kassel (DE)

(21) Appl. No.: 12/157,271

(22) Filed: Jun. 9, 2008

(30) Foreign Application Priority Data
Dec. 14, 2005 (DE) 102005060193.6-52

(51) Int. Cl. A61L 2/16; G01N 31/00

(52) U.S. Cl. 422/5; 422/83

(57) ABSTRACT

The invention relates to a method for the continuous reduction of the odor pollution caused by the waste water in sewers, comprising the following steps: a) determining the actual pollution of the waste water by means of a water sample of the waste water, b) calculating the quantity of odor-reducing agent required to reduce the odor pollution below a defined threshold value, using the degree of pollution, c) adding the defined quantity of odor-reducing agent to the waste water, d) continuously repeating steps a) to c). The aim of the invention is to devise a device and a method of the above type for reliably detecting odor pollution and for reducing it to a minimum using the odor-reducing agents. For this purpose, in step a) the pollution of the waste water is determined by determining the pollution of the water sample with odorous substances.

The present invention relates to a method as set forth in the preamble of the claims 1 and 3 as well as to a device as set forth in the preamble of the claims 11 and 20.

Various wastewater types are discharged into the sewer system so that odors may develop through the discharged wastewater, through microbiological processes or through chemical reactions of various kinds of discharged wastewater. The odors then generated may create considerable nuisance and, as a result thereof, harmful environmental effects in the sense of the Federal Law relative to Emission Protection. Accordingly, the odor emissions are to be minimized, which is usually achieved by reducing odor development.

From the document DE 40 07 064 A1 there is known a device for determining relevant substances in a liquid, said device comprising a gas analysis equipment that is fed with the components to be measured released from the liquid via an extraction tube. Through a gas supply tube, a carrier gas is thereby injected into the flowing liquid, said carrier gas releasing the gas components to be measured which are then supplied to the gas analysis equipment together with the carrier gas through the extraction tube. For this purpose, the extraction tube is connected with an immersion tube projecting into the liquid, said immersion tube enclosing a porous distribution body that is connected to the gas supply tube for supplying the carrier gas in the form of finely distributed gas bubbles, the flowing liquid impinging laterally onto said distribution body.

The company Yara GmbH puts into practice a method for reducing the development of odors in wastewater according to which a sample of wastewater is collected for investigation for hydrogen sulphide (H₂S). The water sample is thereby acidified with HCl to a pH of 4 before air is blown through the water sample for stripping the hydrogen sulfides contained in the water sample. Then, the amount of hydrogen sulfides contained in the stripped air may then be readily acquired by online measurement. Depending on the amount of H₂S contained in the water sample, a certain quantity of an odor reducing means is added to the waste water, upstream thereof. This odor reducing means is permanently added to the waste water so that the development of odors is continuously reduced.

Ascertaining current wastewater contamination by determining the concentration of hydrogen sulfides however has not been found sufficient since, if it is true that an increased concentration of hydrogen sulfides causes odors to develop, the reverse is not permissible. Finally, odor emissions may also have other causes so that missing hydrogen sulphide is not a guarantee for lack of odors.

In urban sewer systems, electronic odor measuring apparatus of WMA Airsense Analysetechnik GmbH or of Altrasens GmbH (Boeker et al: Methodik und Technik der Online-Geruchsmessung aus Gefahrstoffe-Reinhaltung der Luft (Methodology and Technology of Online Odor Measurement for Keeping Air free of Hazardous Substances, 63 (2003) No. 7/8, pages 283-289) are utilized by means of which the odor concentration in the air is measured. If a limit value is exceeded, an odor-reducing agent is added to the wastewater. This measurement is prone to errors since the odor meter must be mounted at a large distance from the actual wastewater on the one side in order to avoid damage to the odor meter and since, on the other side, environmental impacts affect the measurement result, for example when fresh air enters into the sewer system through an opening. Another disadvantage of this odor measurement in the sewer air is that the odorous compounds contained in wastewater are not released to ambient air to the same extent everywhere. Less odorous compounds are for example released at places at which the wastewater flows quietly as compared to places at which the wastewater is turbulent.

All the methods outlined herein above for ascertaining the current wastewater contamination are not capable of reliably ascertaining the odorous compounds really contained in the wastewater. In order to nevertheless reduce odor emissions or development of odors to a bearable extent, more than the actually needed quantity of odor-reducing agent is preventively added to wastewater. Although this allows for usually sufficiently reducing the detected odor concentration, it happens in cases that the odor concentration is not detected and, as a result thereof, is not fought. It is understood that the high utilization of odor-reducing agents also involves high costs.

In view thereof, it is the object of the present invention to provide for a device and a method of the type mentioned herein above that allows for reliably detecting odor concentration and for minimizing the utilization of odor-reducing agents.

As a technical solution to this object, a method having the features of claim 1 or the features of claim 3, and a device having the features of claim 11 or the features of claim 19 are proposed. Advantageous developed implementations of this method and of this device will become apparent from the respective dependent claims.

A method implemented according to this technical teaching and a device configured according to this technical teaching offer the advantage that, by measuring the odorous compounds actually contained in the wastewater instead of, like in prior art, only one single odorous compound, namely hydrogen sulphide, a very precise information on an existing contamination and on the degree of contamination is available and allows for calculating with great accuracy the quantity of odor-reducing agents to be utilized. This precise knowledge regarding the actual contamination, meaning regarding the actually existing amount of odorous compounds renders unnecessary to overdose the odor-reducing agents so that considerable cost savings may be achieved. Another advantage is that through precise knowledge of the actually currently existing odorous compounds odor contamination may be prevented efficiently and reliably. Still another advantage is that, thanks in particular to the device mentioned herein, a fully automated and almost continuous detection of the odorous compounds is achieved so that developing odor contamination may be fought promptly.

It has been found advantageous to rinse the entire device after each measurement, the parts in contact with wastewater being rinsed with fresh water whilst the parts coated with odorous compounds are rinsed with fresh air. This permits to avoid erroneous measurement results.

For reasons of costs, ambient air is used for blowing the odorous compounds away. It has been found advantageous to clean the ambient air with an activated-carbon filter, a mineral filter and/or a pollen filter prior to utilizing it in the reactor and/or to dehumidify the ambient air in order not to distort the measurement results.

In a preferred embodiment, it has been found advantageous to configure the reactor to be a vertically oriented cylinder, more specifically to dimension the reactor so as to spare a freeboard having less than a third of the reactor's height, preferably of less than 10 cm, in order to minimize the air volume in the reactor. The freeboard has the advantage to prevent possibly generated foam from entering the air line or the diverse measuring apparatus.

In another preferred embodiment, the cylindrical reactor has a height-to-diameter ratio of at least 2, preferably of 6. This brings the advantage that the medium to be blown into the reactor, preferably air, must travel a sufficiently long distance through the water sample so that the odorous compounds may be released from the water sample in order to be transported out of the water sample by the gaseous medium.

In another preferred embodiment, it has been found advantageous to provide for a membrane at the bottom of the reactor. This membrane, which preferably acts in only one direction, has for example a number of evenly spaced slots for the air to pass through. This offers the advantage that the gaseous medium to be injected, preferably the air, is distributed evenly over the cross section of the reactor and that the bubbles forming during injection have a defined size so that the odorous compounds are optimally stripped from the water sample.

In still another preferred embodiment, a negative pressure applied in the reactor assists in filling the water sample into the reactor. As a result, the water sample will enter the reactor without great turbulence or swirl. The advantage thereof is that, when filling the water sample into the reactor, only a negligible small amount of odorous compounds may escape from the water sample so that the measurement result obtained thereafter will not be distorted significantly.

Fresh wastewater is permanently pumped from the sewer system into an extraction line for supplying wastewater from the sewer system to the reactor. The water line leading to the reactor and through which the water sample is taken is then connected to this extraction line.

This brings the advantage that the extraction line may be laid ad lib, so that the measuring device may be placed anywhere and needs not be placed in immediate proximity to the sewer system and so that current and unaltered wastewater is still available for measurement.

In a completely different, preferred embodiment, wastewater contamination is determined by determining both the odorous compounds contained in the water sample and the sulphide value in a water sample, an evaluation unit utilizing the two measured values in order to dose the odor-reducing agents. The advantage thereof is that, by measuring both the odorous compounds and the hydrogen sulphide, any possible odor generating sources will be detected and can be fought accordingly.

Further advantages of the method of the invention and of the device of the invention will become apparent in the appended drawings and in the following description of embodiments thereof. Likewise, the invention lies in each and every novel feature or combination of features mentioned above or described herein after. The embodiments discussed herein are merely exemplary in nature and are not intended to limit the scope of the invention in any manner. In said drawings:

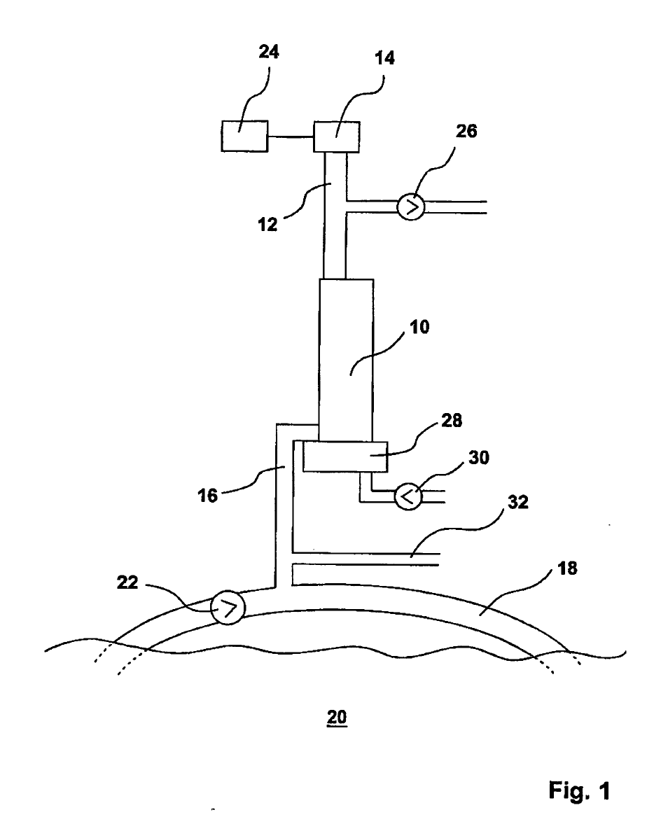

FIG. 1 shows a schematic diagram of a device of the invention for determining the concentration of odorous compounds in a water sample;

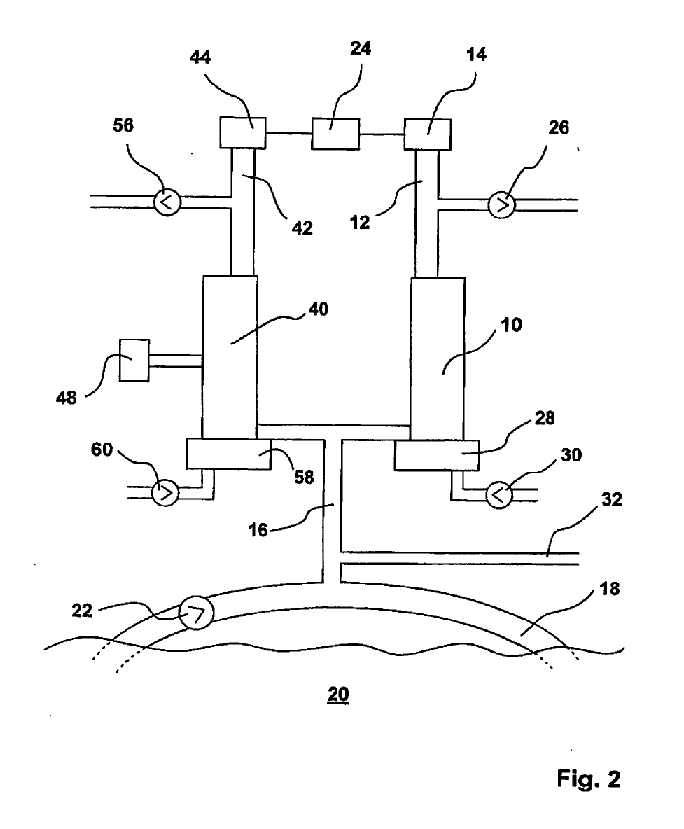

FIG. 2 is a schematic diagram of a device of the invention for determining the contamination of a water sample;

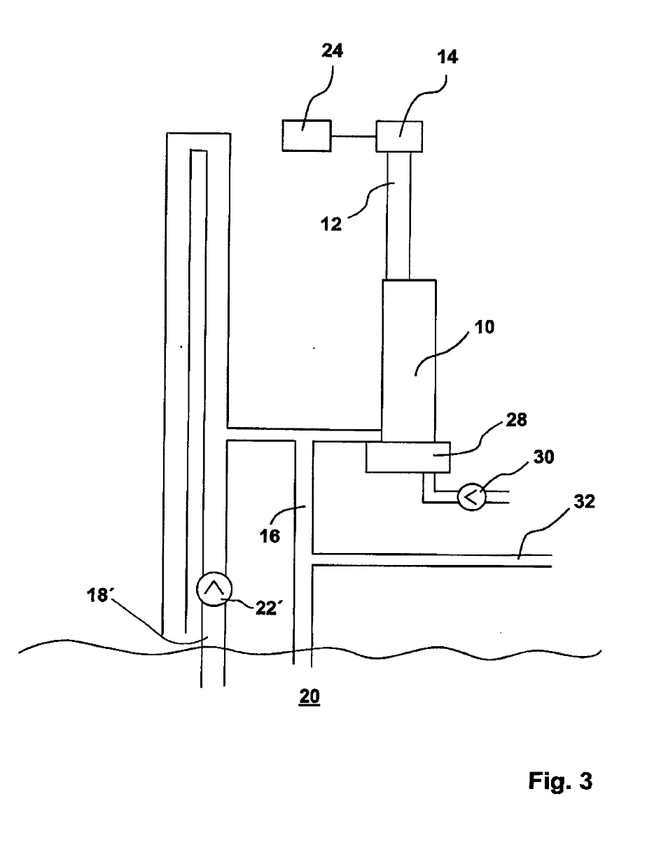

FIG. 3 shows a schematic diagram of an alternative embodiment of a device of the invention for determining the concentration of odorous compounds in a water sample.

FIG. 1 shows an embodiment of a device of the invention for determining the concentration of odorous compounds in a water sample, comprising a cylindrical reactor 10, to the output side of which there is connected an odor meter 14 via an air line 12. On the input side, a water line 16 is connected to the reactor 10, said water line being connected through an extraction line 18 to the wastewater 20 of the sewer system. Wastewater 20 is permanently, meaning either continuously or at regular intervals, pumped into the extraction line 18 by an extraction pump 22. An evaluation unit 24, in which the detected measured values are stored and at need processed, is connected to the odor meter 14.

A negative pressure device 26 for applying a negative pressure to the reactor 10 is connected to the air line 12. This negative pressure device 26 may serve to control the filling of the reactor; this will be explained in closer detail herein after. For filling the reactor 10 with a water sample, a valve that has not been illustrated herein is opened in the water line 16 and negative pressure is applied to the reactor. Pumped wastewater now flows into this partial vacuum through the water line 16 from the extraction pump 22 to the extraction line 18 until the negative pressure device 26 is switched off and the valve in the water line 16 is closed again.

At the bottom of the reactor 10 there is provided a membrane 28 through which air compressed by a compressor 30 may be injected into the reactor 10. The membrane, which is made from an elastomer, has a number of slots that are distributed evenly over the surface and through which the compressed air may pass. The air is thereby distributed evenly over the cross section of the reactor 10 through the membrane 28, or rather through the slots, and the size of the bubbles in the water sample can be adjusted by designing the slots accordingly.

The cylindrical reactor 10 has a height-to-diameter ratio of about 6 and a capacity of about 900 ml. The reactor 10 has a freeboard of about 10 cm so that possibly generated foam is prevented from entering the air line or the diverse measuring apparatus.

Upon completion of measurement, the water sample is evacuated into the extraction line 18 by opening the corresponding valves that have not been illustrated herein, being thereby controlled by the negative pressure device 26. Next, the entire device is cleaned by allowing tap water carried in a cleaning line 32 to flow into the water line 16 and the reactor 10, before it is discharged again through the cleaning line 32. Next, the compressor 30 pumps fresh air into the reactor in order to clean the reactor space, the air line and the odor meter 14.

Herein after, the method for continuously reducing odor contamination of wastewater and the method for determining the concentration of odorous compounds in a water sample will be described herein after in closer detail:

Wastewater from the urban sewer system is permanently circulated through an extraction line 18 by means of the extraction pump 22, not needed wastewater being again discharged into the sewer system. For determining the contamination of the wastewater, a wastewater 20 sample is now supplied to the reactor 10. For this purpose, a corresponding valve is opened in the water line 16 and the negative pressure device 26 is activated so that wastewater flows from the extraction line 18 via the water line 16 into the reactor 10. By virtue of the negative pressure, the water sample can flow quietly and evenly, meaning without greater swirl or turbulence, into the reactor 10. Such type swirls or turbulences might cause odorous compounds from emanating prematurely, which in turn could distort the measurement result. Exactly 900 ml are supplied to the reactor 10. Then, the compressor 30 is activated and fresh air is injected into the reactor 10 through the membrane 28. Through the membrane 28, the fresh air is evenly distributed over the cross section and it enters the reactor 10 with a defined bubble size. Since the reactor 10 has a height six times its width, the injected air travels a sufficient distance within the water sample for the odorous compounds contained in the water sample to be stripped. This means that these odorous compounds escape from the water sample and are evacuated from the reactor 10 together with the air in the air line 12. At the end of the air line 12, the quantity of odorous compounds contained in the air is measured and the measured result is stored in the evaluation unit 24. Over a time period of five minutes, exactly 90 Liters/h of air are blown through the reactor 10 so that the water sample to air ratio is 1 to 100.

In another embodiment that has not been shown herein, the water sample to air ratio may range between 1 to 5 through 1 to 500.

In still another embodiment that has not been illustrated herein, a gas different from air, for example nitrogen, may be used. The reactor may also have another dimension but it should be made certain that the reactor has a height sufficient (at least twice its width) for the air blown therethrough to have enough time to absorb the odorous compounds. After the odorous compounds have been measured, the water sample is again discharged into the extraction line 18 before the entire device is rinsed. For this purpose, fresh water is pumped by the cleaning line 32 through the water line 16 into the reactor 10. After cleaning, the water is again discharged via the water line 16 and the cleaning line 32. Then, the compressor 30 injects fresh air into the reactor so that hardly any odorous compounds remain in the device. Now, the next measurement starts in a cleaned device so that measurement distortions are minimized as a result thereof.

Such a measurement cycle is repeated at short time intervals of about ten minutes so that the odor contamination of the wastewater is almost continuously measured.

The measured values stored in the evaluation unit 24 are transferred to a computing unit for computing the necessary quantity of odor-reducing agent that has not been illustrated herein. Since the entire device is designed for the water-to-air ratio to be 1 to 100, the measured values registered may, in comparison with reference values, give information about the concentration of odorous compounds in the water sample. According to this concentration, a defined quantity of odor-reducing means is to be added to the wastewater via a dosing device that has not been illustrated herein. Since the wastewater contamination is measured continuously, changes in the odor contamination can be detected and eliminated quickly so that resident nuisance may be reliably prevented. Moreover, by determining the current wastewater contamination, the quantity of odor-reducing agents to be utilized may be calculated with great accuracy so that the costs for reducing odor nuisance are reduced as a result thereof.

In another embodiment that has not been illustrated herein the ambient air injected by the compressor 30 is cleaned by means of an activated carbon filter, a mineral filter and/or a pollen filter and possibly dehumidified prior to entering the reactor so that the air used for measurement will not comprise compounds that would distort the measurement.

In FIG. 2, a second embodiment of a device for determining the contamination of a water sample is shown in which, in addition to the device for determining the concentration of odorous compounds in a water sample described in FIG. 1, there is also provided a device for determining the actual hydrogen sulfide concentration of a wastewater sample. This device also comprises a cylindrical reactor 40 that is connected on its output side with a hydrogen sulfide meter 44 via an air line 42. On the input side, the reactor 40 is connected to the extraction line 18 via a water line 16 into which wastewater is permanently pumped from the sewer system through an extraction pump 22.

The values obtained during measurement are transferred by the hydrogen sulfide meter 44 to the evaluation unit 24 where they are stored and at need further processed.

At the input, a membrane 58 is provided at the reactor 40 through which a compressor 60 injects 90 standard liters/h of air into the reactor 40. The reactor has a capacity of 900 ml so that the ratio water sample to injected air is 1 to 100. The ratio height of the reactor 40 to diameter of the reactor 40 is about 6 so that the air to be injected for stripping the hydrogen sulfides has to travel a sufficient distance in the water sample for the hydrogen sulfides to be absorbed.

A dosing pump 48 by means of which HCl may be added to the water sample is connected to the reactor 40. The amount of HCl added to the water sample is such that the entire water sample will have a pH of no more than 4.

Here also, a negative pressure device 56 for controlling the filling of the reactor with water is connected to the air line 42. Once the reactor 40 is filled with the water sample, the water sample is acidified by adding HCl. At the input side, a membrane 58 and a compressor 60 through which the 90 liters/h of ambient air are supplied to the reactor 40 are provided at the reactor 40. This membrane is configured analogous to membrane 28.

Through the membrane 58; the air is thereby evenly distributed over the cross section of the reactor 40 and comprises a defined bubble size. Upon completion of measurement, the water sample is discharged to the extraction line 18 through the water line 16. The entire device is again cleaned after measurement, tap water being transported to the reactor 40 through the cleaning line 32 and being also evacuated therefrom through this cleaning line 32. Fresh air is then supplied to the reactor 40 through the compressor 60 in order to clean the reactor space and the air line as well as the hydrogen sulfide meter 4.

The method of determining wastewater contamination by means of a device for determining the odorous compound concentration in a water sample and of a device for determining the hydrogen sulfide concentration in the water sample is described as follows:

On the one side, a water sample is supplied from the extraction line 18 to the reactor 10 and the odorous compound concentration in the water sample is determined according to the method described referring to FIG. 1, the measured values obtained being stored in the evaluation unit 24. On the other side, a second water sample is supplied from the extraction line 18 through the water line 16 into the reactor 40. This occurs by means of negative pressure for the water sample not to experience unnecessary turbulence or swirl which might cause part of the hydrogen sulfide to emanate prematurely, thus distorting the measurement result. The water sample has a volume of 900 ml and fills the reactor 40 so as to spare a freeboard of 10 cm at the top.

Next, the current pH of the water sample is measured and the water sample is acidified by adding HCl through the dosing pump for the water sample to have a pH of no more than 4. Then, the compressor 60 injects 90 liters of air through the membrane 58 into the reactor 40. This standard air extracts the hydrogen sulfides contained in the water sample and transports them to the hydrogen sulfide meter 44 which then stores the measured valued obtained in the evaluation unit 24.

Finally, the water sample is discharged into the extraction line 18 through the water line 16 and the reactor 40 is rinsed with tap water that is again discharged through the cleaning line 32. To finish, the compressor 60 pumps fresh air into the reactor 40 and into the air line 42 in order to also clean it and eliminate all the remaining odor particles, hydrogen sulfides or other contaminations.

This measurement may be repeated every 2 minutes or so, so that the concentration of hydrogen sulfide in the wastewater may be measured almost continuously.

The values stored in the evaluation unit 24 and relating to the odorous compound concentration in the water sample and relating to the hydrogen sulfide concentration in the water sample are transferred to a computing unit which then computes the appropriate dosage of odor-reducing agents to be added to the wastewater.

FIG. 3 shows an alternative embodiment of a device for determining the odorous compound concentration in a water sample. As contrasted with the first embodiment shown in FIG. 1, the extraction line 18′ is here configured to be a riser duct into which the extraction pump 22′ delivers the wastewater. As a result, the wastewater needed for the water sample enters the reactor 10 by virtue of the pressure generated by the extraction pump 22′ as soon as the corresponding valves are open. In this embodiment, the water sample needs no longer be drawn into the reactor by means of negative pressure. It has been found advantageous to lay the extraction line 18′ so that it extends beyond the reactor for the wastewater to completely fill the reactor 10 when the valves are open. The advantage thereof is that the water sample is pumped into the reactor 10 and is not drawn into it, this reducing the undesirable escape of odorous compounds prior to actual measurement.

As an odor-reducing agent, H₂O₂ or another oxidant may be utilized. The odorous compounds may also be degraded by precipitation, for example with iron. Oxygen may also be added to the wastewater in order to preventively reduce odors. A combination of some or all of the means mentioned is also possible.

The device of the invention or the method of the invention may not only find application in sewer systems, this device or this method may also be utilized in industrial applications. In this case, solid or liquid residues could for example be measured and be provided with odor-reducing agents that must be stored in open air or that must be discharged into rivers, lakes or into the sewer system.

In another embodiment that has not been illustrated herein, a foam retaining device is provided in the reactor for reducing or preventing foam formation when the water sample is filled into the reactor. This foam retaining device may for example be formed from gauze or from a grid and is preferably mounted in the center of the reactor or in the region of the final fill level.

### LIST OF NUMERALS

10 reactor
12 air line
14 odor meter
16 water line
18 extraction line
20 wastewater
22 extraction pump
24 evaluation unit
26 negative pressure device
28 membrane
30 compressor
32 cleaning line
40 reactor
42 air line
44 hydrogen sulphide meter
48 dosing pump
56 negative pressure device
58 membrane
60 compressor

1. A method for continuously reducing odor contamination of wastewater in sewer systems, involving the following steps:
a.) ascertaining the current wastewater contamination by means of a wastewater sample,
b.) computing the necessary quantity of an odor-reducing agent as a function of the degree of contamination for lowering the odor contamination below a fixed limit value,
c.) adding the defined quantity of the odor-reducing agent to the wastewater,
d.) continuously repeating the steps a.) to c.),
characterized in
that wastewater contamination is ascertained in step a.) by determining the odorous compound concentration in the water sample.

2. The method as set forth in claim 1,
characterized in
that in step a.) wastewater contamination is ascertained by determining the odorous compound concentration in the water sample and by determining the sulfide concentration in the water sample.

3. A method for determining the odorous compound concentration in a water sample, characterized by the following steps:
aa) supplying a defined quantity of the water in the form of a water sample to a reactor,
ab) blowing the odorous compounds out of the water sample by means of a gaseous medium, preferably by means of ambient air,
ac) leading the blown-out medium together with the odorous compounds into an odor meter,
ad) measuring the quantity of odorous compounds by means of the odor meter and transferring the measurement result to an evaluation unit,
ae) evacuating the water sample from the reactor.

4. The method as set forth in claim 3,
characterized in
that next to step ae) the following step is performed:
af) rinsing the reactor with fresh water and/or rinsing the lines with fresh air.

5. The method as set forth in, claim 3 characterized in
that the ratio water sample to gaseous medium is between 1 to 5 and 1 to 500, and preferably is 1 to 100, more particularly that the water sample ranges between 600 ml and 1200 ml, and preferably is 900 ml, between 60 liters/h and 120 liters/h, preferably 90 liters/h of the medium being utilized for blowing the odor compounds out.

6. The method as set forth in claim 3,
characterized in
that the ambient air is cleaned by means of an activated carbon filter, a mineral filter and/or a pollen filter prior to entering the reactor.

7. The method as set forth in claim 3, characterized in
that the ambient air is dehumidified prior to entering the reactor.

8. The method as set forth in claim 3, characterized in
that the water sample is drawn into the reactor by means of negative pressure.

9. The method as set forth in claim 1, characterized in
that the wastewater sample is freshly taken.

10. The method as set forth in claim 1, characterized in
that wastewater is permanently pumped from the sewer system into an extraction line (18) and that the water sample is taken out of the extraction line (18).

11. A device for ascertaining the current wastewater contamination with a reactor (10) for receiving the water sample to which there is connected a device for blowing the odor compounds out of a water sample by means of a gaseous medium, with an odor meter (14) for measuring the odorous compounds contained in the blown out medium, said odor meter (14) being connected to the output side of the reactor (10) via an air line (12).

12. The device as set forth in claim 11,
characterized in
that the reactor (10) is configured to be a vertically oriented cylinder.

13. The device as set forth in claim 11, characterized in
that the reactor (10) is dimensioned so as to spare a freeboard having less than a third of the reactor's height after the water sample has been filled into it.

14. The device as set forth in claim 13,
characterized in
that the freeboard is less than 10 cm.

15. The device as set forth claim 11, characterized in
that a membrane (28) is provided on the input side of the reactor (10).

16. The device as set forth in claim 11, characterized in
that the cylindrical reactor (10) has a height-to-diameter ratio of at least 2, preferably of 6.

17. The device as set forth in claim 11, characterized by
a device for determining the sulfide concentration in a water sample and by an evaluation unit (24), said evaluation unit (24) taking into consideration both the current sulphide concentration and the current degree of odorous compound concentration in the water sample for computing the actual contamination.

18. The device as set forth in claim 11, characterized in
that a water line (16) is mounted to the input side of the reactor (10, 40), said water line being in operable communication with the wastewater (20) and that a negative pressure device (26, 56) for applying negative pressure to the reactor (10, 40) is connected to the output side of the reactor (10, 40).

19. The device as set forth in claim 18,
characterized in
that the water line (16) is connected to an extraction line (18) through which wastewater (20) is permanently pumped.

20. A device for continuously reducing odour contamination of wastewater in the sewer system, with a computing unit for computing the quantity of odor-reducing agent needed and with a dosing system,
characterized by
a device for determining the odorous compound concentration in a water sample or by a device for ascertaining the current contamination as set forth in claim 11.
